(19) United States
(12) Patent Application Publication
(10) Pub. No.: US 2026/0311829 A1
(43) Pub. Date: Oct. 8, 2026

(54) AUTOMATED RISK MODEL PROCESSING AND MULTIDIMENSIONAL PROVIDER MATCHING ARCHITECTURE

(71) Applicant: Evernorth Strategic Development, Inc., St. Louis, MO (US)

(72) Inventors: Priya Needs, Holly Springs, NC (US); Carol Quinlan, Montvale, NJ (US); Andrew Long, Austin, TX (US); Beth Taylor, Wethersfield, CT (US); Jason A. Short, Murfreesboro, TN (US); Shauna Mooney, Richmond, VA (US)

(21) Appl. No.: 19/715,186

(22) Filed: Jun. 22, 2026

Related U.S. Application Data
(63) Continuation of application No. 18/204,834, filed on Jun. 1, 2023.
(60) Provisional application No. 63/348,167, filed on Jun. 2, 2022.

(51) Int. Cl. G16H 10/60; G06Q 40/08; G16H 20/10

(52) U.S. Cl. CPC G16H 10/60; G06Q 40/08; G16H 20/10

(57) ABSTRACT

A method for automated entity field correction includes receiving one or more target health conditions, obtaining a set of multiple patient entries, stored patient data, stored claims data and stored prescription data, and determining an eligibility status for each patient entry according to specified eligibility criteria, indicative of the patient entry being eligible for targeted outreach regarding the target health condition(s). For each patient entry in an eligible subset, the method includes determining an exclusion status for the patient entry according to specified exclusion criteria, indicative of the patient entry being excluded from targeted outreach regarding the target health condition(s). The method includes accessing stored provider data, and for each patient entry in the non-excluded subset, determining a provider match for the patient entry according to at least a portion of the stored provider data, and transmitting the provider match to a computing device associated with the patient entry.

### CROSS-REFERENCE TO RELATED APPLICATION

This application claims the benefit of U.S. Provisional Application No. 63/348,167, filed on Jun. 2, 2022. The present application is a continuation of U.S. patent application Ser. No. 18/204,834, filed Jun. 1, 2023. The entire disclosures of the applications referenced above are incorporated herein by reference.

### FIELD

The present disclosure relates to automated risk model processing and multidimensional provider matching architecture.

### BACKGROUND

Patients that receive ongoing behavioral outpatient care may have significantly lower medical and pharmacy costs compared to patients that do not receive ongoing behavioral outpatient care, which demonstrates the value of behavioral health care for patients and providers. Improvements have been made in advancing behavioral health, such as reducing the stigma, reframing the conversation to highlight the connection between physical and mental health, and improving access to treatment by rapidly growing behavior health care networks and virtual care services to meet people where they are.

However, a low percentage of people newly diagnosed with a behavioral health condition access care with a behavioral health provider, indicating a large opportunity to help more people improve their health and well-being. This need may be particularly important for people with comorbidities, with even greater total health care costs associated with patients having comorbid behavioral and physical health conditions.

In some cases, multiple behavioral outpatient sessions, such as counseling or group therapy, may reduce medical costs, which provides an opportunity to identify people who stop treatment too early and help them find care to meet their needs. As demand for mental health continues to grow, a renewed effort may guide people to the behavioral health care they need to help them live healthier, more productive lives.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A method for automated entity field correction includes receiving one or more target health conditions, obtaining a set of multiple patient entries, stored patient data, stored claims data and stored prescription data, and for each patient entry in the set of multiple patient entries, the set of multiple patient entries, stored patient data, stored claims data and stored prescription data each stored in one or more databases. The method includes accessing at least a portion of the stored patient data, stored claims data and stored prescription data corresponding to the patient entry, determining an eligibility status for the patient entry according to specified eligibility criteria and the accessed portion of the stored patient data, stored claims data, and stored prescription data, wherein the determined eligibility is indicative of the patient entry being eligible for targeted outreach regarding the one or more target health conditions, and in response to the patient entry having an eligible status, assigning the patient entry to an eligible subset of the set of multiple patient entries. For each patient entry in the eligible subset, the method includes determining an exclusion status for the patient entry according to specified exclusion criteria and the accessed portion of the stored patient data, stored claims data, and stored prescription data, wherein the exclusion status is indicative of the patient entry being excluded from targeted outreach regarding the one or more target health conditions, and in response to the patient entry having a non-excluded status, assigning the patient entry to a non-excluded subset of the set of multiple patient entries. The method includes accessing stored provider data, and for each patient entry in the non-excluded subset, determining a provider match for the patient entry according to at least a portion of the stored provider data, and transmitting the provider match to a computing device associated with the patient entry, to display the provider match on a user interface for selection by a user.

In other features, the method includes training a machine learning model with historical feature vector inputs to generate a provider match prediction output, wherein determining the provider match includes supplying the at least a portion of the stored provider data to the trained machine learning model to generate the provider match prediction output, and assigning the provider match prediction output as the provider match. In other features, the specified eligibility criteria includes a condition that the patient entry is experiencing the one or more target health conditions.

In other features, the specified eligibility criteria includes a condition that the patient entry has not exceeded a specified threshold of treatment visits within a specified time period. In other features, the specified eligibility criteria includes a condition that the patient entry has a specified coverage type for at least a specified time period.

In other features, determining a provider match includes searching for a provider data update using at least one of multiple application programming interfaces (APIs). In other features, a first one of the multiple APIs is configured to search a first provider data source, and a second one of the multiple APIs is configured to search a second provider data source other than the first provider data source.

In other features, displaying the provider match includes displaying a set of multiple provider matches in response to a first preference input from the user, and displaying a subset of the set of multiple provider matches in response to a second preference input from the user. In other features, the specified eligibility criteria includes a condition that the patient entry does not have a specified acute behavioral condition or acute medical condition within a specified time period. In other features, the specified exclusion criteria includes a condition that the patient entry does not have a total number or specified chronic medical conditions over a specified threshold value.

A computer system includes memory hardware configured to store computer-executable instructions, and processor hardware configured to execute the instructions. The instructions include receiving one or more target health conditions, obtaining a set of multiple patient entries, stored patient data, stored claims data and stored prescription data, the set of multiple patient entries, stored patient data, stored claims data and stored prescription data each stored in one or more databases of the memory hardware, and for each patient entry in the set of multiple patient entries, accessing at least a portion of the stored patient data, stored claims data and stored prescription data corresponding to the patient entry, determining an eligibility status for the patient entry according to specified eligibility criteria and the accessed portion of the stored patient data, stored claims data, and stored prescription data, wherein the determined eligibility is indicative of the patient entry being eligible for targeted outreach regarding the one or more target health conditions, and in response to the patient entry having an eligible status, assigning the patient entry to an eligible subset of the set of multiple patient entries. For each patient entry in the eligible subset, the instructions include determining an exclusion status for the patient entry according to specified exclusion criteria and the accessed portion of the stored patient data, stored claims data, and stored prescription data, wherein the exclusion status is indicative of the patient entry being excluded from targeted outreach regarding the one or more target health conditions, and in response to the patient entry having a non-excluded status, assigning the patient entry to a non-excluded subset of the set of multiple patient entries. The instructions include accessing stored provider data, and for each patient entry in the non-excluded subset, determining a provider match for the patient entry according to at least a portion of the stored provider data, and displaying the provider match on a user interface for selection by a user.

In other features, the instructions further include training a machine learning model with historical feature vector inputs to generate a provider match prediction output, wherein determining the provider match includes supplying the at least a portion of the stored provider data to the trained machine learning model to generate the provider match prediction output, and assigning the provider match prediction output as the provider match. In other features, the specified eligibility criteria includes a condition that the patient entry is experiencing the one or more target health conditions.

In other features, the specified eligibility criteria includes a condition that the patient entry has not exceeded a specified threshold of treatment visits within a specified time period. In other features, the specified eligibility criteria includes a condition that the patient entry has a specified coverage type for at least a specified time period.

In other features, determining a provider match includes searching for a provider data update using at least one of multiple application programming interfaces (APIs). In other features, a first one of the multiple APIs is configured to search a first provider data source, and a second one of the multiple APIs is configured to search a second provider data source other than the first provider data source.

In other features, the method includes receiving a selection of the provider match via the user interface of the computing device, and in response to receiving the selection of the provider match, transmitting the selection of the provider match to a server to update a database entry associated with the patient entry.

In other features, displaying the provider match includes displaying a set of multiple provider matches in response to a first preference input from the user, and displaying a subset of the set of multiple provider matches in response to a second preference input from the user. In other features, the specified eligibility criteria includes a condition that the patient entry does not have a specified acute behavioral condition or acute medical condition within a specified time period. In other features, the specified exclusion criteria includes a condition that the patient entry does not have a total number or specified chronic medical conditions over a specified threshold value.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a functional block diagram of an example system for automated risk model processing and multidimensional provider matching.

FIG. 5 is a message sequence chart illustrating example interactions between components of the system of FIG. 4.

FIG. 6 is a flowchart depicting an example process for automated risk model processing and multidimensional provider matching.

FIG. 7 is a flowchart depicting an example process for determining eligible patient entries.

FIG. 8 is a flowchart depicting an example process for determining patient entry exclusions.

FIG. 9 is a flowchart depicting an example process for provider matching.

FIGS. 10A and 10B are graphical representations of example recurrent neural networks for automated multidimensional provider matching.

FIG. 11 is a graphical representation of layers of an example long short-term memory (LSTM) machine learning model.

FIG. 12 is a flowchart illustrating an example process for training a machine learning model.

FIGS. 13A, 13B and 13C are example interfaces for receiving user input regarding provider matching preferences.

FIGS. 14A, 14B and 14C are example interfaces for displaying provider matches, receiving a provider selection, and scheduling an appointment with a provider.

FIG. 15 is a flowchart depicting an example method for generating a ranked list of search results using a patient provider matching system.

In the drawings, reference numbers may be reused to identify similar and/or identical elements.

### DETAILED DESCRIPTION

### HIGH-VOLUME PHARMACY

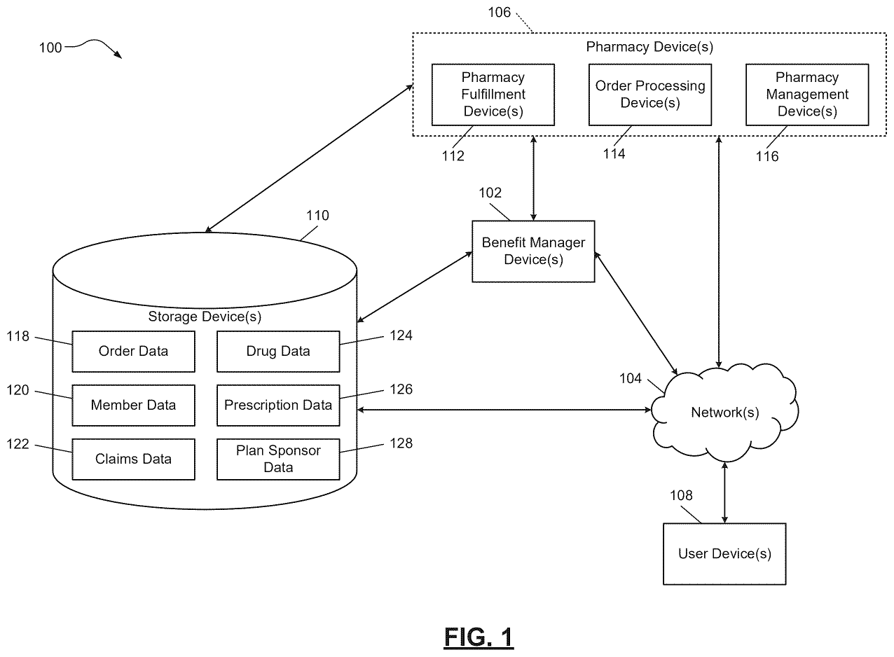

FIG. 1 is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104.

The system 100 may also include one or more user device(s) 108. A user, such as a pharmacist, patient, data analyst, health plan administrator, etc., may access the benefit manager device 102 or the pharmacy device 106 using the user device 108. The user device 108 may be a desktop computer, a laptop computer, a tablet, a smartphone, etc.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in a storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug is successfully adjudicated. The aforementioned adjudication operations generally occur before the copayment is received and the prescription drug is dispensed. However, in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfillment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally, or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

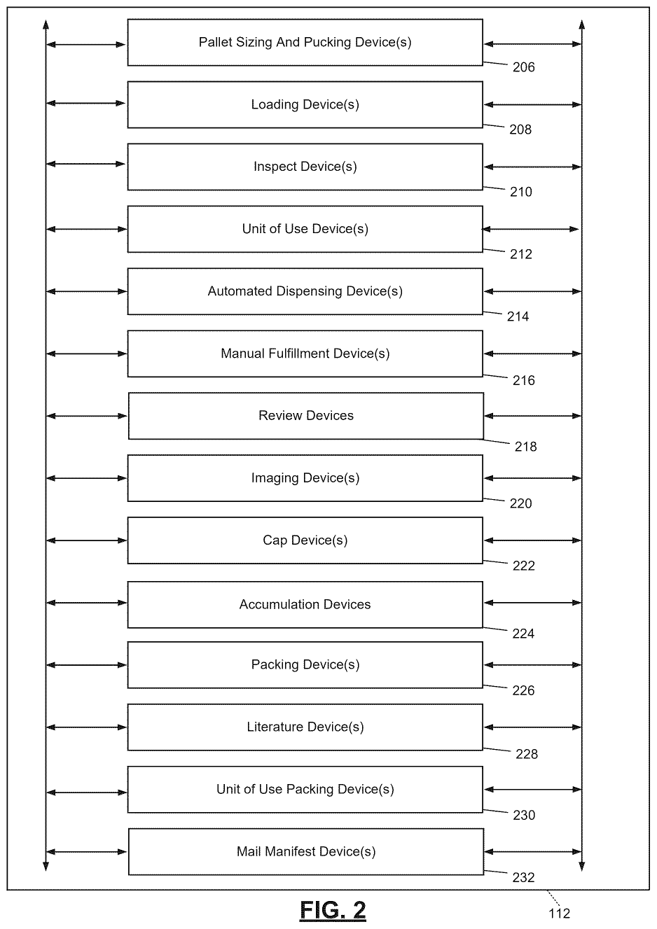

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

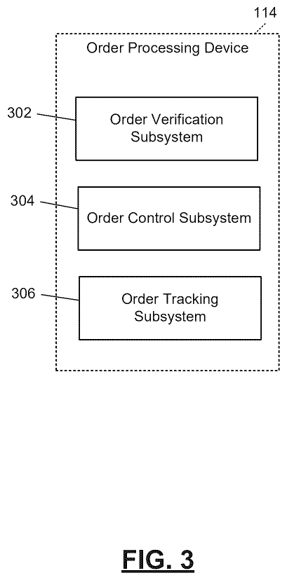

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may be comprised of order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

### AUTOMATED RISK MODEL AND PROVIDER MATCHING SYSTEM

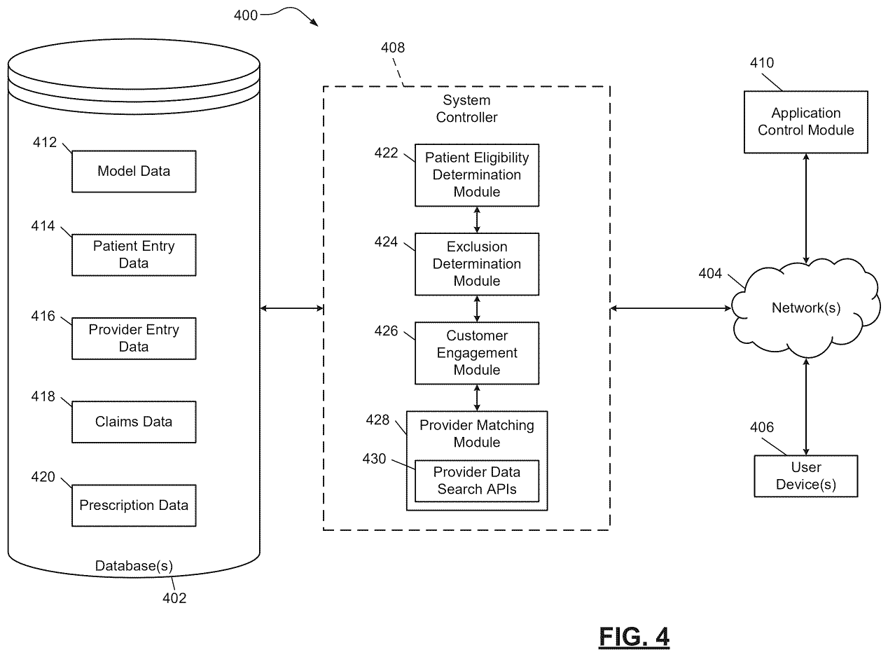

FIG. 4 is a functional block diagram of an example system 400 for automated risk model processing and multidimensional provider matching, which includes a database 402. While the system 400 is generally described as being deployed in a computer network system, the database 402 and/or components of the system 400 may otherwise be deployed (for example, as a standalone computer setup). The system 400 may include a desktop computer, a laptop computer, a tablet, a smartphone, etc.

As shown in FIG. 4, the database 402 stores model data 412, patient entry data 414, provider entry data 416, claims data 418, and prescription data 420. In various implementations, the database 402 may store other types of data as well. The model data 412, patient entry data 414, provider entry data 416, claims data 418, and prescription data 420 may be located in different physical memories within the database 402, such as different random access memory (RAM), read-only memory (ROM), a non-volatile hard disk or flash memory, etc. In some implementations, the model data 412, patient entry data 414, provider entry data 416, claims data 418, and prescription data 420 may be located in the same memory (such as in different address ranges of the same memory). In various implementations, the model data 412, patient entry data 414, provider entry data 416, claims data 418, and prescription data 420 may each be stored as structured or unstructured data in any suitable type of data store.

The model data 412 may include any suitable data automatically filtering patients for targeted behavioral health outreach (e.g., such as patient eligibility data, patient exclusion data, etc.), any suitable data for automatically matching providers with patients according to provider preference values associated with each patient, for training one or more machine learning models (such as training a machine learning model to identify provider matches based on provider preference values associated with a patient), etc. For example, the model data 412 may include historical feature vector inputs that are used to train one or more machine learning models to generate a prediction output, such as a prediction of a best provider match. The historical feature vector inputs may include the historical data structures which are specific to multiple historical database entities (such as multiple historical patient and provider matches).

In various implementations, users may run, train, etc. a model by accessing the system controller 408 via the user device 406. The user device 406 may include any suitable user device for displaying text and receiving input from a user, including a desktop computer, a laptop computer, a tablet, a smartphone, etc. In various implementations, the user device 406 may access the database 402 or the system controller 408 directly, or may access the database 402 or the system controller 408 through one or more networks 404. Example networks may include a wireless network, a local area network (LAN), the Internet, a cellular network, etc.

The system controller 408 may include one or more modules for automated risk model processing and multidimensional provider matching. For example, FIG. 4 illustrates a patient eligibility determination module 422, an exclusion determination module 424, a customer engagement module 426, and a provider matching module 428. The provider matching module 428 may include or more provider search application programming interfaces (APIs) 430.

The patient eligibility determination module 422 may identify patient entries (e.g., patient entities stored in one or more databases corresponding to customers of a health care service payer, a provider, employees of a business, etc.), which may be eligible for targeted outreach for one or more specified medical conditions (such as anxiety, depression, or other behavioral health conditions).

The exclusion determination module 424 may remove patient entries from a subset of determined eligible patient entries, according to specified exclusion criteria. For example, the exclusion determination module 424 may remove patient entries associated with acute medical conditions, a threshold number of chronic health conditions, specified prescription drug utilizations, etc.

The customer engagement module 426 may facilitate outreach to patients within the non-excluded subset of eligible patient entries. For example, the customer engagement module 426 may schedule outreach messages (such as emails or texts), may facilitate download of a customer engagement mobile application, may schedule a human intervention such as a customer care phone call or outreach by a physician, etc. The customer engagement module 426 may interact with the application control module 410 to suggest downloading of an application to non-excluded eligible patients, monitor engagement with the mobile application, transmit messages to a patient via the mobile application, etc.

The customer engagement module 426 may interact with the provider matching module 428 to identify provider entries (e.g., provider entities stored in one or more databases corresponding to health care providers that may provide care for a patient), that match with a patient entry according to specified provider match criteria. For example, the provider data search APIs 430 may search various provider data sources, such as provider databases, provider websites, provider information storage systems, periodical news articles, etc., to obtain information about provider entries to determine whether het prover entry would be a good match for a patient or customer.

Referring back to the database 402, the patient entry data 414 may include any suitable data records of patients and associated field values, such as a patient name, address, date of birth, phone number, employment status, medical and prescription drug insurance coverage, social media information, and so on. The provider entry data 416 may include any suitable data records for providers and associated field values, such as a provider physician name, a health care specialization of the provider, a provider address, a provider identifier, a provider phone number, provider demographic information, provider ratings and reviews, and so on.

The claims data 418 may include any suitable data records of patients for medical or behavioral treatment, such as past medical or behavioral history, medical or behavioral treatment visits, medical or behavioral procedure code, etc. The prescription data 420 may include any suitable data records for prescription drug utilization for a patient, such as dates of prescription drug fills, prescription drug codes, etc. In various implementations, more or less (or other) data may be stored in the database 402. The database 402 may be considered as a record database where the patient entry data 414 includes multiple patient entity data structures, and the provider entry data 416 includes multiple provider data structures.

In various example embodiments, any suitable hardware and/or software components may be used to implement one or more features of the system 400 for automated risk model processing and multidimensional provider matching. For example, a structured analytic engine such as OpenSAE maybe used to process patient data from a corporate warehouse database, via Hadoop.

Extraction criteria may be defined to determine what data is obtained for patient selection algorithms. A customer interaction manager (CIM) may be used to automate campaign management for contacting patients, such as via emails, with appropriate contact preferences and privacy checks. A patient device application may be used for patient interaction, such as displaying patient provider match information and obtaining patient input as described further below.

A care decision management engine (CDM) may be configured to interpret patient survey information and other clinical inputs, make decisions about care paths for the patient, and display recommendations on a user interface. An elevate survey platform may be used to obtain patient inputs.

Online content providers may provide behavioral health information to a patient, through an application of the system 400. The behavioral health information may include articles, media, videos, etc. In some example embodiments, provider information may be received from a curated provider feed, etc.

In various implementations, provider matching may be performed in any suitable manner, such as an elastic search via an API. Patient data may be stored in any suitable manner, such as a longitudinal patient record. A patient application may interface with a patient behavioral history document generator, to generate PDFs based on data associated with the patient, etc.

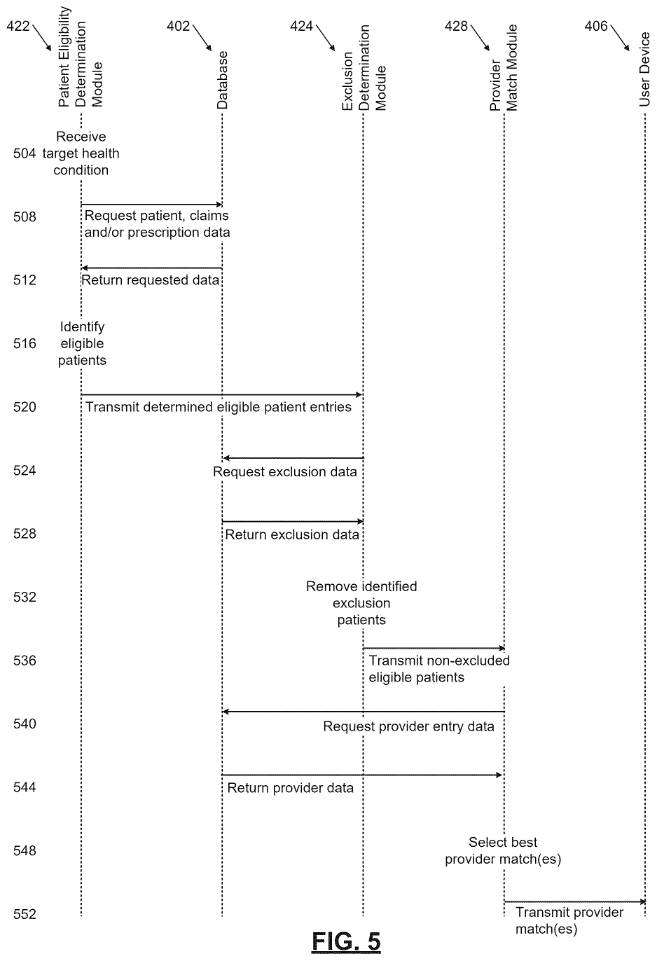

FIG. 5 is a message sequence chart illustrating example interactions between the database 402, the patient eligibility determination module 422, the exclusion determination module 424, the provider matching module 428, and the user device 406. At line 504, the patient eligibility determination module 422 receives a target health condition. For example, the patient eligibility determination module 422 may identify patient entries associated with depression and/or anxiety, or any other suitable behavioral health condition or medical condition.

At line 508, the patient eligibility determination module 422 requests patient entry data (such as the patient entry data 414 in FIG. 4), claims data (such as the claims data 418), prescription data (such as the prescription data 420), etc., from the database 402. The database 402 returns the requested data to the patient eligibility determination module 422 at line 512.

At line 516, the patient eligibility determination module 422 identifies eligible patient entries (e.g., eligible patients obtained from the patient entry data 414). The eligible patient entries may be determined according to eligible patient entry specified criteria. The patient eligibility determination module 422 may generate a subset of patient entries according to the specified criteria, by removing patient entries that do not satisfy the specified criteria.

The patient eligibility determination module 422 then transmits the determined eligible patient entities (e.g., the subset of eligible patients) to the exclusion determination module 424 at line 520. The exclusion determination module 424 requests exclusion data at line 524. The database 402 returns the requested exclusion data at line 528.

At line 532, the exclusion determination module 424 removes exclusion patients. For example, the exclusion determination module 424 may remove any patient entries from the subset of determined eligible patients if the patient entries meet specified exclusion criteria, such as having an acute health condition, having multiple chronic health conditions, having a specified prescription drug utilization, a patient already attending multiple behavior health treatment sessions within a specified recent time period (e.g., within the last month, within the last year), etc.

The exclusion determination module 424 transmits the non-excluded eligible patient entries to the provider matching module 428 at line 536. The provider matching module 428 then requests provider data from the database 402, at line 540. The database 402 returns the requested provider entry data at line 544.

The provider matching module 428 selects the best provider matches for each patient, at line 548. For example, the provider matching module 428 may use one or more APIs to access information about provider entries (which may be accessed from various provider data sources), and the provider matching module 428 may determine a best provider match based on specified provider matching criteria. At line 552, the provider matching module 428 transmits the provider match(es) to another computing device, to a database, to computer display, to a mobile application, etc.

In various implementations, the system 500 may facilitate assisting patients in getting access to behavioral health services. This may assist patients with their quality of life, and unaddressed behavioral conditions have also been associated with higher medical costs. Helping more patients may address medical costs which can benefit patients, employers, providers, payers, etc.

With the reduction in stigma on mental health in society, the number of providers and the ability to service patients is a constrained resource. Some behavioral health providers may favor treating patients that they can make a connection with, because providers may not find as much satisfaction with treating non-compliant patients, and providers may not desire to constantly seek new patients to replace patients that abandon their care without benefit.

Even when people engage with behavioral health services, they can drive increased costs to all parties if not effectively facilitated. For example, if patients have a bad provider experience and discontinue treatment early, there are expenses with paying for these services without realizing benefits. And many health plans cap or govern the number and/or frequency of approved provider visits, so if treatment goes on indefinitely without material benefit, this also drives up costs given that investments being made to address mental health are not yielding value to the patient. There is a large population of people who do not engage or address their mental health issues, in addition to patients that engage but then quickly disengage before receiving any benefit. Example systems described herein may use automated modeling, data processing, provider matching algorithms, etc., to address both of these populations (as well as others).

### AUTOMATED RISK MODELING AND PROVIDER MATCHING PROCESS

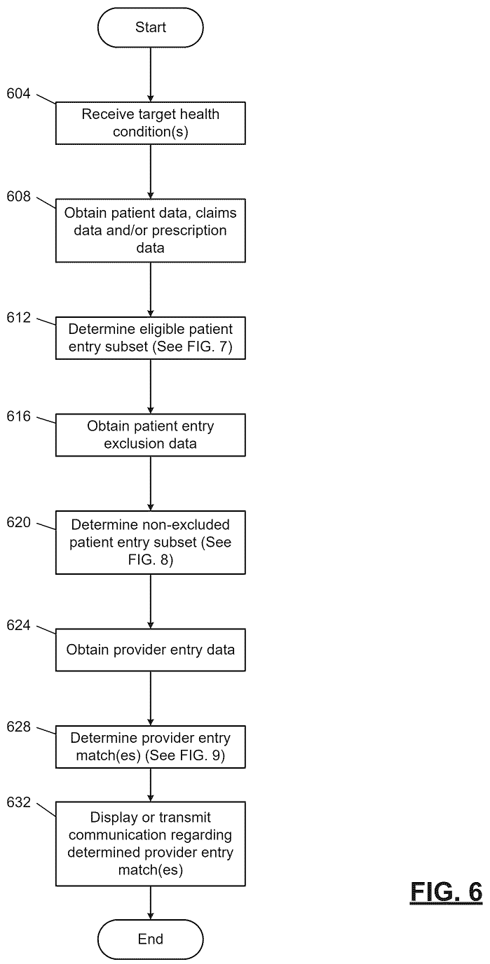

FIG. 6 illustrates an example process for automated risk model processing and multidimensional provider matching, which may be performed by, for example, one or more modules of the system controller 408. Control begins at 604 by receiving target health condition(s). For example, control may receive target behavioral health conditions such as depression and/or anxiety, or any other suitable behavioral health or medical conditions.

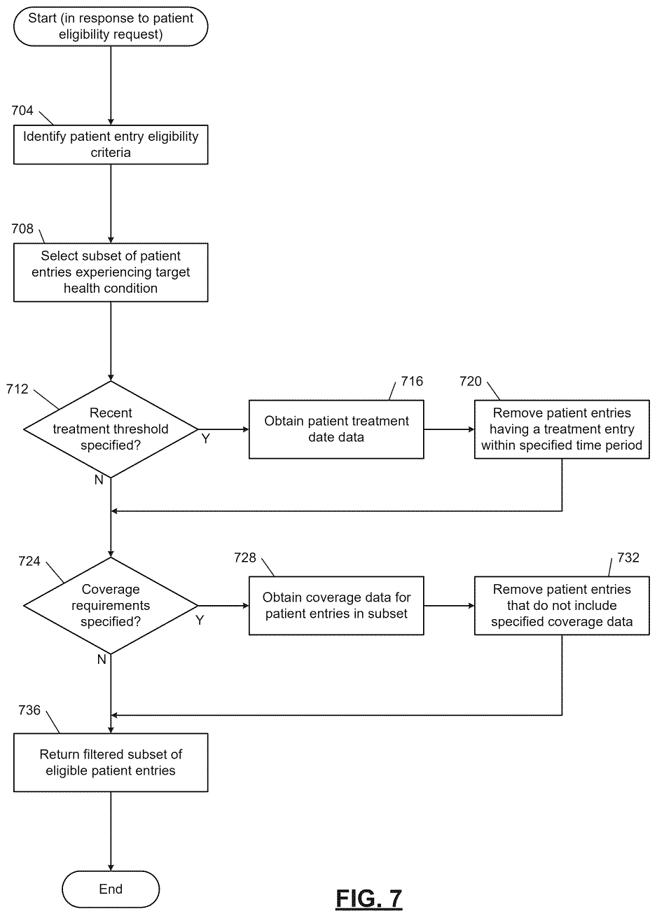

At 608, control obtains patient data, claims data, prescription data, etc. Control then determines an eligible patient entry subset at 612. An example process for determining the eligible patient entry subset is described in further detail below with reference to FIG. 7.

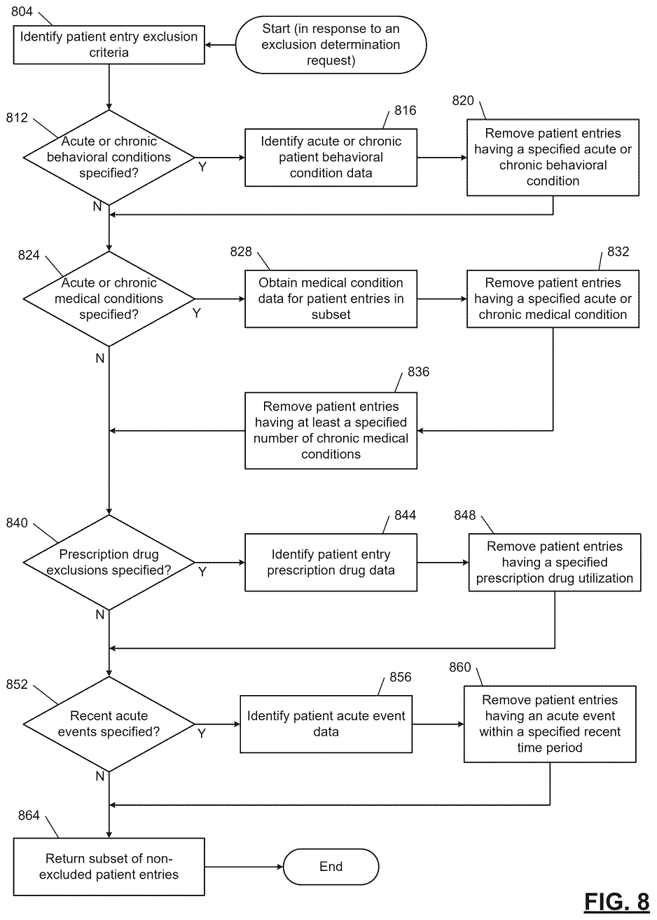

At 616, control obtains patient entry exclusion data. Control then determines a non-excluded patient entry subset at 620. An example process for determining the non-excluded patient entry subset is described further below with reference to FIG. 8.

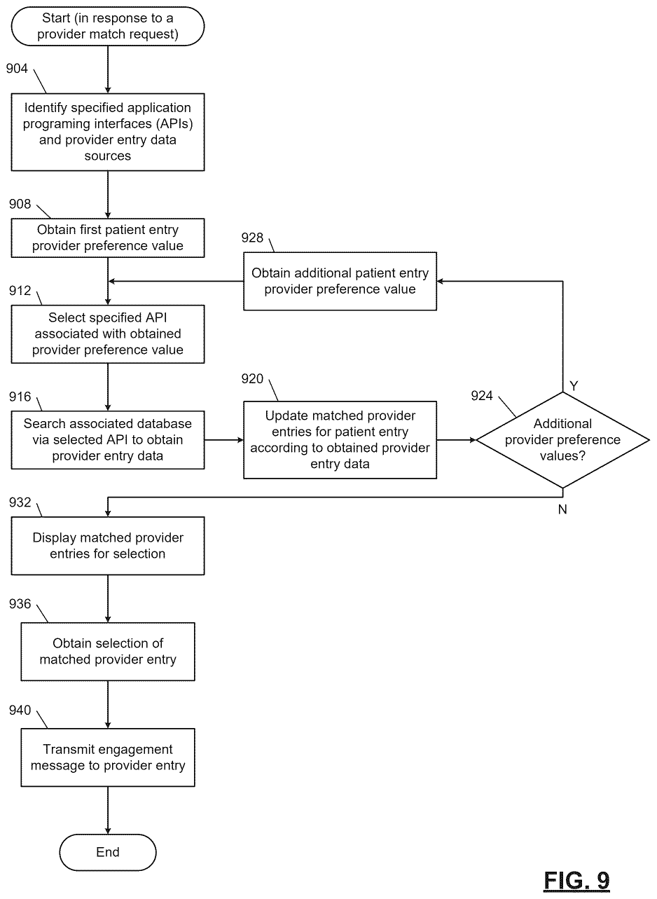

Control obtains provider entry data at 624. At 628, control determines one or more provider entry matches (e.g., best matches, optimal matches, minimum threshold matches, etc.). An example process for determining provider entry match(es) is described further below with reference to FIG. 9. At 632, control displays, transmits, etc., communication regarding the determined provider entry match(es).

In various implementations, control may monitor one or more metrics for patient engagement. For example, control may monitor a number of patients targeted by outreach efforts that engaged in outpatient care (such as a behavioral health treatment session), within a specified time period (e.g., within one month, within three months, etc.). Control may monitor a length of time between a provider match and a first treatment appointment for a patient, a percentage of targeted outreach patients that remained connected to care for a specified number of visits (e.g., at least two visits, at least three visits, at least four visits, etc.), a percentage of patients that switched providers after or a number of provider switches, whether patients would recommend matched providers or the overall system to others, how many targeted patients end up on escalation or emergency pathways, etc.

In various implementations, control may monitor engagement in an outreach application by monitoring a number of patients (e.g., customers) that complete an assessment, that review content, that leverage a provider match feature, that schedule an appointment, etc. Control may monitor customers (e.g., patients) that have outpatient visits after engaging with an outreach application, an amount of time from being matched with a provider to visiting a provider, etc. Control may monitor total cost of care reductions for customers that use the outreach application and provider matching features, how many customers have a visit with a provider or stay with a provider, how customers rate their experiences with the outreach application and provider matching, etc.

FIG. 7 illustrates an example process for determining eligibility of a patient entry. The process may start in response to a request for a patient eligibility determination, such as at block 612 of FIG. 6. At 704, control identifies patient entry eligibility criteria. At 708, control selects a subset of patient entries experiencing a target health condition, such as depression, anxiety, etc.

In various implementations, target health conditions may include specified clinical criteria such as low acuity depression, low acuity anxiety, other suitable behavioral or non-behavioral health conditions, etc. Target health diagnoses of patients may be determined by, for example, looking at diagnosis codes (e.g., ICD-10 codes) associated with the target health condition in patient medical or behavioral records, searching for prescription fills of medications associated with target health conditions (such as antidepressant medications or antianxiety medications), etc. In various implementations, patients may be determined for eligibility based on medical claims only, based on behavioral claims only, based on pharmaceutical claims only, based on a combination of medical claims, behavioral claims and pharmaceutical claims, etc. For example, a diagnosis of depression and/or anxiety on a medical or behavioral claim, or a fill of antianxiety or antidepressant medication, may be indicative that a customer has an anxiety or depression condition. Example medication fills that may indicate a diagnosis of depression and/or anxiety may include, but are not limited to antidepressants (e.g., monoamine oxidase inhibitors, selective serotonin reuptake inhibitors, tricyclic antidepressants, alpha-2 receptor antagonists, norepinephrine and dopamine reuptake inhibitors, NMDA receptor antagonists, etc.), antianxiety medications (e.g., benzodiazepines, sedatives, tricyclic ADP, etc.), etc.

At 712, control determines whether a recent treatment threshold is specified. For example, the patient eligibility criteria may specify that only patients having less than three (in-person or virtual) behavioral outpatient health visits within the last two months are eligible for targeted outreach. In some cases, medical and pharmaceutical costs may be reduced when a newly diagnosed patient receives, for example, three or more behavioral outpatient visits within the first year (or more or less).

If control determines at 712 that the recent treatment threshold is included in the patient eligibility criteria, control proceeds to 716 to obtain patient treatment data. For example, control may access medical or behavioral records to determine when each patient entry had a session for treatment (such as a session with a behavioral health provider).

Control then proceeds to 720 to remove patient entries having a treatment record within a specified time period. For example, patient entries having three or more treatments (e.g., outpatient treatments) within the last two months may be removed from the subset of patients for targeted outreach. In various implementations, more or fewer treatment sessions (e.g., telehealth sessions) or visits may be specified as a threshold, more or less than two months may be specified as a time threshold (such as the last six months or the last year), etc. In various implementations, control may only include patient entries that do not have any previous behavioral outpatient treatment visits or sessions.

After removing the patient entries exceeding the specified treatment threshold at 720, or if control determines at 712 that the optional recent treatment threshold has not been set for use in patient eligibility determination criteria, control proceeds to 724 to determine whether coverage requirements are specified.

For example, targeted outreach may be provided only to patients having specified insurance coverage (e.g., for at least one month, for at least six months, for at least one year, etc.), only patients registered with specified applications or services, only patients who are employees of specified organizations, etc. In various implementations, control may include only patients that have a sufficient period of data available for screening (e.g., at least two months of medical records data, at least six months of medical records data, etc.). A minimum threshold of prior coverage may provide better accuracy regarding the acuity level of patient health conditions and the utilization of behavioral health treatments. If control determines at 724 that coverage requirements are specified, control proceeds to 728 to obtain coverage data for patient entities in the subset.

In various implementations, suitable eligibility criteria may include age (e.g., over 18 years old), whether the patient is registered with a specified application that implements provider matching, whether a patient is in a location with a sufficient number of providers in the area, whether the patient is the main contact on an insurance plan, whether the patient is a member of a specified health insurance plan type, whether the patient is of a specified gender, etc. Optional use of patient age, gender, etc., may be selected based on the type of outreach intervention or health condition targeted.

Coverage data may be obtained by, for example, accessing insurance databases associated with patients, accessing application or service registrations, accessing employment rosters, etc. Control then removes patient entries that do not include the specified coverage data at 732. After removing the patient entries that do not meet the specified coverage requirements, or if control determines at 724 that optional coverage requirements have not been included as criteria for patient eligibility, control proceeds to 736 to return the filtered subset of eligible patient entries.

In various implementations, any suitable optional exclusion criteria may be specified, such as customers with an authorization or claim for a current medical hospitalization, a current behavioral hospitalization, or a current intensive outpatient treatment, customers with an emergency department visit in the last six months (or more or less time) with a behavioral health diagnosis, customers with two or more medical hospitalizations in the last twelve months (or more or less), customers with one or more psychological or behavioral hospitalizations or intensive outpatient treatments in the last twelve months (or more or less), customers currently receiving hospice or palliative care, customers with five or more chronic conditions (or more or less), customers having a fill for an opioid related prescription drug (e.g., for at least ten days or more or less), customers with a fill for ADHD medication, customers with a fill for antipsychotic meds, etc.

In various implementations, control may obtain additional patient information for making eligibility and exclusion determinations, such as chronic back pain (e.g., a lower back pain diagnosis in the past year or more or less), lower back pain or spine surgery with a time period of ninety days (or more or less), weight related diagnoses, insomnia diagnoses, cardiometabolic conditions, a login to a registered service within the last ninety days (or more or less), a recent search by the patient for a therapist or psychiatrist (or other provider), use of other digital provider search tools or ancillary providers, customers engaged in care management or coaching, etc.

FIG. 8 illustrates an example process for determining exclusions for patient entries. The process may start in response to a request for exclusion determinations, such as at block 620 of FIG. 6. At 804, control identifies patient entry exclusion criteria, such as acute medical conditions, a specified number of chronic health conditions, specified prescription drug utilization, etc.

At 812, control determines whether acute or chronic behavioral conditions have been specified. If so, control proceeds to 816 to identify acute or chronic patient behavioral data. For example, control may access medical and behavioral claims data to identify patient entries associated with acute or chronic behavioral health conditions. At 820, control may remove patient entries having specified acute or chronic behavioral conditions. For example, high level of care utilization is indicative that a patient may have high acuity behavioral needs that may require more intensive treatment than outpatient visits.

In various implementations, control may optionally exclude patients having a recent or current behavioral health condition that requires a high level of care, such as autism, bipolar disorder, conduct disorder, court ordered treatment, eating disorders, history of domestic violence or sexual assault, homicide, neurocognitive disorders, personality disorders, psychosis, schizophrenia, self-harm, substance use, suicide, traumatic brain injury, etc., but example embodiments are not limited thereto. Control may identify conditions using diagnosis or procedure codes in medical claims history, such as ICD-10 codes, CPT codes, etc. Other example exclusion criteria for higher levels of behavioral care utilization may include customers with at least one psychiatric inpatient or intensive outpatient treatment in the last 12 months, a residential stay, a partial hospitalization, applied behavior analysis, transcranial magnetic stimulation, etc.

Control may use any suitable lookback period for identifying patient acute behavioral health conditions, such as the last six months, the last year, the last two years, all claims history, etc. Different time periods may be applied to different conditions. In various implementations, a physician or administrator may identify and assign various diagnosis codes as being associated with target health conditions, acute behavioral conditions, acute medical conditions, chronic medical conditions, etc., for use in identifying or excluding eligible patient entries for targeted outreach.

After removing the patient entries having acute behavioral conditions, or if control determines at 812 that acute or chronic behavioral conditions have not been specified as optional exclusion criteria, control proceeds to 824 to determine whether acute or chronic medical conditions have been specified. If so, control proceeds to 828 to obtain medical condition data for patient entries in the subset.

Control removes patient entries having a specified acute or chronic medical condition at 832. For example, control may optionally remove patient entries having a recent or current claims history for Alzheimer's, amputation, blindness, cognitive impairment, dementia, fertility treatment, HIV, kidney disease, liver disease, metastatic cancer, multiple sclerosis, seizure, transplant, etc., but example embodiments are not limited thereto. Control may use any suitable lookback period for identifying patient acute behavioral health conditions, such as the last six months, the last year, the last two years, all claims history, etc. Different time periods may be applied to different conditions. In various implementations, control may optionally exclude patients for targeted outreach that are in hospice. For example, high level of medical care utilization, hospice and palliative care, etc., may be indicative that a patient may has high acuity medical needs that may require more intensive treatment than outpatient visits. In various implementations, customers with recent medical inpatient or emergency room visits may be excluded.

Control then removes patient entries having at least a specified number of chronic medical conditions at 836. For example, control may remove patient entries having at least two chronic medical conditions, at least five chronic medical conditions, or more or less chronic conditions. Examine chronic conditions may include coronary artery disease, congestive heart failure, chronic obstructive pulmonary disease, diabetes mellitus, low back pain, osteoarthrosis, oncology, weight complications, peripheral artery disease, chronic kidney disease, end stage renal disease, etc.

In this approach, patient entries may be removed for either having a specified acute medical condition, or a threshold number of chronic medical conditions. For example, a high level of chronic conditions may be indicative that a patient has higher acuity medical needs that may require more treatment than outpatient visits. The number of chronic conditions for a threshold may be adjusted based on a target outreach intervention, a target medical condition, etc.

After removing the patient entries having acute medical condition or a threshold number of chronic medical conditions, (or determining that acute or chronic medical conditions have not been specified as optional exclusions criteria at 824), control proceeds to 840 to determine whether prescription drug exclusions have been specified. If so, control proceeds to 844 to identify patient entry prescription drug data.

Control removes patient entries having a specified drug utilization at 848. For example, patients that are using specified prescription drugs may have health conditions that are not good candidates for targeted outreach. For example, control may exclude patients having fills for prescription drugs such as antipsychotics (e.g., phenothiazines, dopamine antagonists, butyrophenones, thioxanthenes, etc.), opioids, and stimulants (e.g., TX for ADHD, central nervous system stimulants, adrenergics, etc.). In some cases, antipsychotics, etc. may be indicative that a patient has severe mental illness requiring more than outpatient visits.

After removing patients having the specified prescription drug utilization, or after determining at 840 that prescription drug utilization has not been specified as optional exclusion criteria, control proceeds to 852 to determine whether a patient has experienced a recent acute event. For example, patients who have experienced an acute event (e.g., inpatient utilization, hospice, etc.), within a specified time period (such as the last six months, the last year, etc.), may be excluded from a target group for outpatient behavioral health outreach.

If control determines at 852 that recent acute events have been specified as exclusion criteria, control proceeds to 856 to identify patient acute event data. Control then removes patient entries having an acute event within a specified recent time period at 860. After removing the patient entries at 860, or determining that recent acute events have not been specified as exclusion criteria at 852, control proceeds to 864 to return a subset of non-excluded patient entries.

FIG. 9 illustrates an example process for determining a provider match. The process may start in response to a provider match request, such as at block 628 in FIG. 9. Control begins at 904 by identifying specified application programming interfaces (APIs) and provider entry data sources.

Control then obtains first patient entry provider preference values at 908. The patient entry provider preference values may be obtained using any suitable approach, such as a questionnaire for patients, receiving patient input, identifying demographic and location data of a patient, identifying medical history of a patient, etc.

At 912, control selects a specified API associated with the obtained preference value. For example, control may select an API for searching locations of providers, may select an API for searching another database or data source indicating providers that match with a specified medical condition of the patient, may select a different API for searching providers based on demographic information of providers, etc.

At 920, control updates matched provider entries for a patient entry according to the obtained provider entry data. For example, control may select only a subset of providers within the same state as the patient, within the same city, etc. Control may select only a subset of providers that match treatment specializations that match with a condition of the patient, only providers with sufficient ratings or reviews, only providers that match demographic or background experience preferences of the patient, etc.

At 924, control determines whether there are additional provider preference values. For example, in various implementations a patient may provide a first input (such as location), and have a first subset of providers displayed that match the location. The patient may then provide another preference such as in-network coverage, and control may use another API to search coverage data sources to display only providers matching both the location and in-network coverage preferences of the patient.

Patient preferences may be used to continuously narrow down the subset of matched providers, optionally with a new API search being performed with respect to each new preference criteria. For example, a patient may specify a preferred age of a provider, a preferred rating for a provider, a preferred sex of a provider, a preferred educational background for a provider, etc., and each preference input may trigger another API search call to successively narrow down the subset of providers displayed to the patient (e.g., the subset of matched providers displayed on a mobile application, computer screen, etc. for a patient, as an interactive way for patients to narrow down their preferences and matched providers).

If a patient reaches a point where no providers match all criteria, or no desired providers match all criteria, a patient may back up a level, change preferences, etc., in order to provide a greater number of matched providers (such as by removing a preferences or making a preference less restrictive). In various implementations, control may provide a suggestion to the patient of an optional preference to remove or update to generate a greater number of provider matches (such as increasing the distance of provider locations, looking at out-of-network providers, searching broader treatment specializations, etc.).

If control determines at 924 that additional provider preference values are present or waiting to be input, control obtains an additional patient entry provider preference value at 928. Control then selects a specified API associated with the additionally obtained preference value at 912 and proceeds to 916 to search an associated database according to the newly obtained preference value at 916.

Once control determines at 924 that no more provider preference values are present, or all provider preference values have been searched and applied, control proceeds to 932 to display a matched provider entries for selection. As mentioned above, control may display matched provider entries for a user interactively. In other embodiments, a set of matched provider entries may be displayed to a user at the end of a provider matching process.

At 936, control obtains a selection of a matched provider entry. For example, a user may select one of the matched provider that they would like to contact for possible treatment. At 940, control transmits an engagement message to the provider entry. This may include, but is not limited to, sending an email to the provider, sending a text message to the provider, opening up a communication channel via a mobile application, initiating a call or video chat with the provider, etc.

In various implementations, the provider matching may perform a federated search across multiple sites with multiple APIs. For example, there may not be a single database having all information about all providers, and the multiple APIs may be used to obtain desired information about providers from multiple sources (such as provider websites, provider review and rating sites, databases of provider insurance associations, etc.). In an example, the use of a federated search allows the provider matching system to access new databases by adding an API while the prior APIs can still be used. The inputs from the user device can be parsed, normalized and ranked to generate the APIs. In an example embodiment, a model, e.g., a predictive model as described herein, can be used to perform at least one of the parsing, normalization and ranking of the inputs for a provider match function.

The federated search may be considered as a multidimensional search that leverages multiple data sources or categories (e.g., dimensions) in order to improve provider matching with patients. For example, provider matching may match providers based on efficiency of providers or how often the providers lead to successful patient outcomes, behavioral or personality factors of providers (e.g., based on reviews of provider mannerisms, whether a provider has a Type A personality, whether a provider has a kind bedside manner, whether the provider has a heart of a teacher, etc.), where the provider is located, what is the provider's specialty or level of expertise (e.g., based on educational background, length of practice, depth of knowledge, etc.), cost of providers, reviews of providers, etc. Social triggers may be obtained by scanning personal profiles of providers, such as by mining via natural language processing (NLP). In various implementations, different weights may be applied to different provider factors based on, e.g., user preferences, etc. These weights may be stored in a table in memory. The table can be accessed applied after the inputs from the patient are parsed and normalized. The weights can rank the inputs for provider matching.

The provider matching application may ask a patient question in an interactive format, in order to narrow down provider matches for the patient. For example, in a behavioral health use case, after a patient logs into an application it may search for all providers in a patient's state (which may be pulled from databases, etc.).

The application may ask questions to the patient about the patient's condition, and use the response information to filter the matched providers. In various implementations, each patient input response may trigger the application to initiate a new API call to pull more information from outside the application, such as searching data sources to identify, e.g., only providers within a specified health insurance coverage plan, only providers with a specified age or gender, etc. In an example, the provider matching may not wait for the entire, complete entry of the patient preference inputs to begin generating APIs to retrieve data from at least one database. The provider matching may parse the preference input as it is entered and begin the searching calls, using e.g., the APIs, before all patient inputs are received. When the returned data is received, then the provider match may rank, e.g., weight, the return data to establish one or more provider data records that match the patient inquiry.

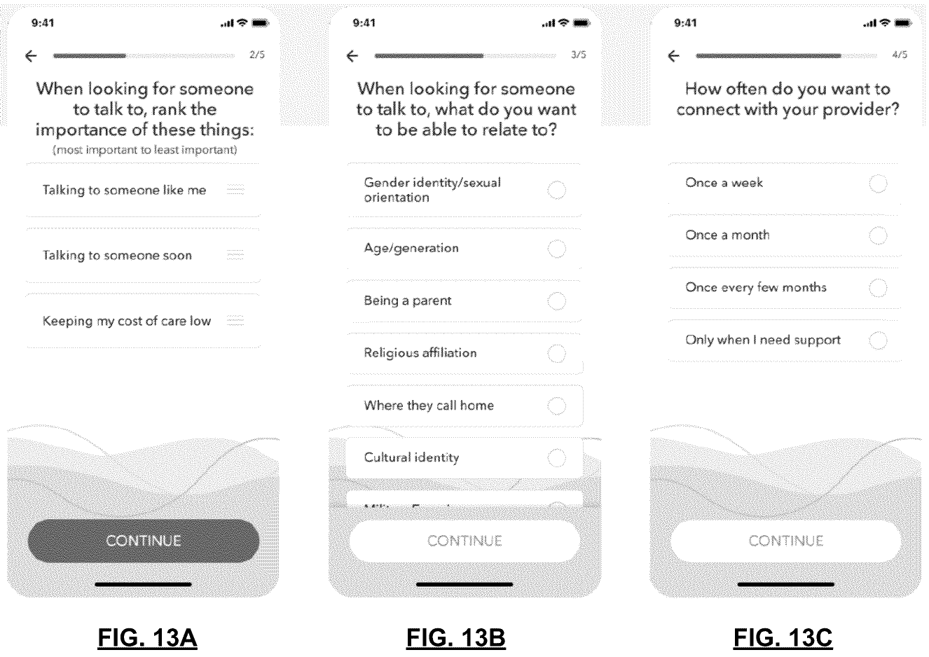

FIG. 13A illustrates an example interface for receiving preference inputs from a user. The example of FIG. 13A asks the user how important various factors are to a user searching for a provider, such as talking to someone similar to the user, talking to a provider that has availability soon, and keeping cost of care low.

FIG. 13B illustrates an example interface asking a patient about preferential characteristics of a provider, such as gender identity/sexual orientation, age/generation, being a parent, religious affiliation, hometown, cultural identity, etc. FIG. 13C illustrates an example interface asking a patient about how often they would like to connect with a provider, such as once a week, once a month, once every few months, only when they need support, etc.

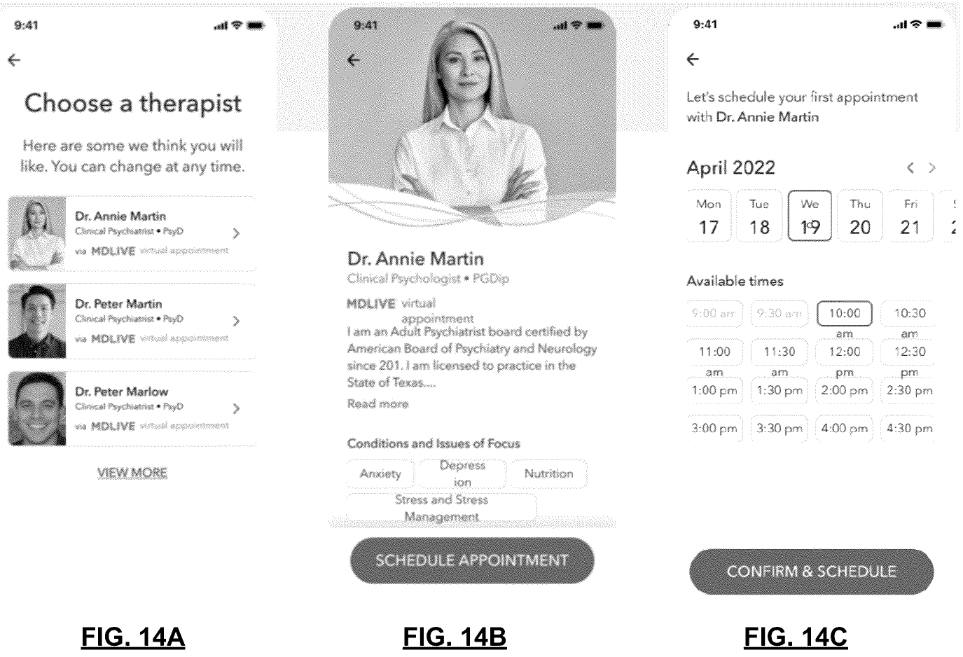

The responses input by the user may be used to identify providers having a best match with the patient preferences. For example, FIG. 14A illustrates an example interface for choosing between multiple therapists having the best matches to the patient preferences. The application may provide an option for the user to select one of the therapists as shown in the example interface of FIG. 14B, and to schedule an appointment with the therapist directly through the application as shown in the example interface of FIG. 14C.

In various implementations, the application may not be dependent on searching all patient preference parameters at one time. For example, the application may continue to filter provider match results each time a patient answers another preference question, in order to create an improved user experience. The application can, in some examples, parse the inputs from the patient device in real time and launch database queries before an entire entry input is made by the patient. If there are not providers meeting a specific preference, such that the number of provider matches is zero, the application may intelligently ask the patient if they want to back up to remove some preference requirements (such as going out of plan), look at other preference factors as more important, etc. In another example embodiment, the provider matching system can use a model to adjust the ranking of a null set preference data return based on prior adjustments to a null set by the provider match for a similar patient.

In various implementations, provider biography data may be used for patient matching, such as providers having similar educational backgrounds, by comparing to patient biography data or preferences. As another example, social media information about providers may be obtained and used as another factor for provider matching.

In various implementations, the provider matching information may work in cooperation with other patient eligibility determination and exclusion determination processes described herein. For example, provider matching information or patient preference information may be fed back into the eligibility determination process to improve filtering of eligible patients for targeted outreach and provider matching.

In some cases, information about patients gathered during the patient eligibility and exclusion determination processes may be used for patient matching. For example, information about patient medical history may be used to automatically filter providers having a specialization in the area of the patient's health conditions.

Patient engagement may be monitored to determine how long certain providers stay matched with patients. A rate at which patients drop providers may be used to improve future patient matching determinations. For example, patient matching improvements may be used to reduce the number of customers that drop providers, or to provide outreach to a customer population that otherwise may not engage providers to receive treatment.

The provider matching system may operate by receiving a provider match query, e.g., from a device at a first location. The provider match system can extract terms from the provider match query by parsing the terms from the provider match query on a term-by-term basis. The term-by-term basis can be performed in real time while the information is input, e.g., in a free form text input or through the use of structured queries. The system can determine if any inputs in the provider match query are relevant to a provider match query by comparing the inputs to known inputs stored in a table, using a model to identify the relevant inputs, or the like. The system can also normalize the inputs before identifying the relevance to a provider match function. The system can further track the databases that relate to specific ones of the inputs and generate APIs to the specific databases, which can be at a second location remote from the processing circuitry identifying, parsing and normalizing the inputs. In some cases, there may be missing terms in the inputs and the normalization function may fill the missing slots with the relevant terms such that the API can request the relevant data from the specified database. The queries in the APIs or the returned data can be assigned a weight to assist with ranking the provider match result. In an example, the resulting provider match must meet or exceed a minimum provider matching threshold, which can be predetermined, in order for any individual provider match to be output, e.g., displayed, from the provider match system.

A patient application, such as the example screens illustrated in FIGS. 13A-C and 14A-C, may provide various functionality, such as onboarding of new patients, content display, surveys, provider matching, provider scheduling, provider concierge, provider visits, etc.

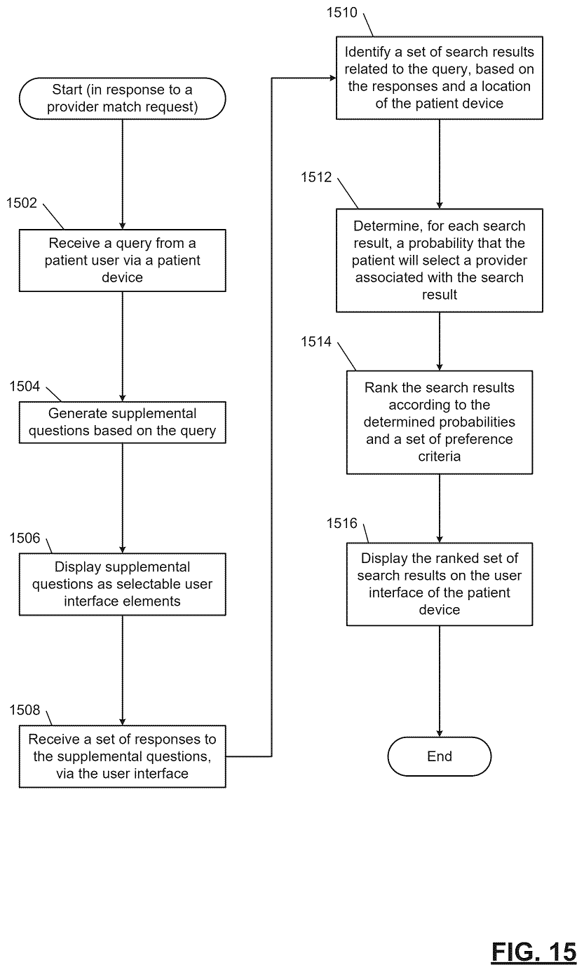

FIG. 15 is a flowchart depicting an example method for generating a ranked list of search results using a patient provider matching system. At 1502, control receives a query from a patient user on a mobile device or other user device.

Based on the query, control generates a set of supplemental questions at 1504. For example, the user interface screens illustrated in FIGS. 13A-C and 14A-C may be used to receive patient queries and to display supplemental questions to gather more information. The supplemental questions may be adapted based on prior input from the patient user.

The supplemental questions may be displayed as selectable user interface elements on a graphical user interface of the user device, at 1506. For example, the supplemental questions may be displayed as selectable user interface elements similar to buttons or input fields displayed in the example screens of FIGS. 13A-C and 14A-C.

At 1508, control receives a set of responses to the set of supplemental questions, via user input to the graphical user interface of the user device. At 1510, control identifies a set of search results related to the query, based on the set of responses to the supplemental questions (and/or initial patient questions) and a location of the user device.

For each search result in the identified set of search results, control may determine, at 1512, a probability that the patient user will select a provider associated with the search result based on a set of patient data associated with the patient user, and a set of provider data associated with the provider. Control may rank the search results based on the determined probability and a set of preference criteria, at 1514.

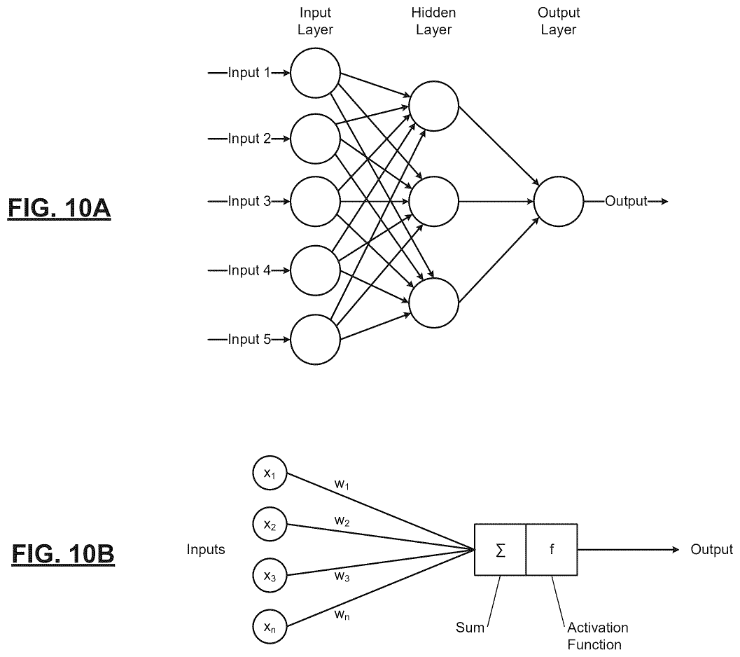

For example, a probability may be determined using a predictive model, such as the example machine learning models described herein with reference to FIGS. 10-12. At 1516, control transmits the ranked set of search results to the user device, to display the ranked set of search results on a graphical user interface of the user device.

### MACHINE LEARNING MODELS

In various implementations, machine learning models may be used to improve provider matching characteristics. Examples of various types of machine learning models that may be used for provider matching are described below and illustrated in FIGS. 10-12, but example embodiments are not limited thereto.

In various implementations, historical provider selections by patients may be used to generate training data for machine learning models. For example, longitudinal engagement of providers by patients may be tracked, which may include multiple providers. If a patient switches between multiple providers over a short period of time, and then stays with another provider for a longer period of time, this may indicate that the last provider works well with the patient and is a good match. This sequence of good and poor provider matches for the patient may be used as training data to train a machine learning model to predict provider matches for patients based on their preferences.

In various implementations, concerns regarding patient non-use or ineffective use of behavioral health services, and their effects on providers and medical costs (such as those described herein), may be addressed by example provider matching algorithms described herein to facilitate improved or optimized matching of patients and providers. For example, novel machine learning techniques may be used to identify unengaged patient populations (e.g., via example techniques described herein), to provide targeted outreach campaigns and effectively match them with appropriate behavioral health service providers.

Improved provider matching can drive better outcomes for patients, increased satisfaction for all parties, and reduce medical costs. The improved provider matching, such as by using example techniques described herein, may help providers by steering patients to them that match their personality, expertise, and treatment approaches, thereby improving provider satisfaction as well. In addition, the improved provider matching may drive more providers to want to offer their services via example system platforms described herein, enabling behavioral health services for more patients.

FIGS. 10A and 10B show an example of a recurrent neural network used to generate models such as those described above, using machine learning techniques. Machine learning is a method used to devise complex models and algorithms that lend themselves to prediction (for example, patient and provider matching predictions). The models generated using machine learning, such as those described above, can produce reliable, repeatable decisions and results, and uncover hidden insights through learning from historical relationships and trends in the data.

The purpose of using the recurrent neural-network-based model, and training the model using machine learning as described above, may be to directly predict dependent variables without casting relationships between the variables into mathematical form. The neural network model includes a large number of virtual neurons operating in parallel and arranged in layers. The first layer is the input layer and receives raw input data. Each successive layer modifies outputs from a preceding layer and sends them to a next layer. The last layer is the output layer and produces output of the system.

FIG. 10A shows a fully connected neural network, where each neuron in a given layer is connected to each neuron in a next layer. In the input layer, each input node is associated with a numerical value, which can be any real number. In each layer, each connection that departs from an input node has a weight associated with it, which can also be any real number (see FIG. 10B). In the input layer, the number of neurons equals number of features (columns) in a dataset. The output layer may have multiple continuous outputs.

The layers between the input and output layers are hidden layers. The number of hidden layers can be one or more (one hidden layer may be sufficient for most applications). A neural network with no hidden layers can represent linear separable functions or decisions. A neural network with one hidden layer can perform continuous mapping from one finite space to another. A neural network with two hidden layers can approximate any smooth mapping to any accuracy.

The number of neurons can be optimized. At the beginning of training, a network configuration is more likely to have excess nodes. Some of the nodes may be removed from the network during training that would not noticeably affect network performance. For example, nodes with weights approaching zero after training can be removed (this process is called pruning). The number of neurons can cause under-fitting (inability to adequately capture signals in dataset) or over-fitting (insufficient information to train all neurons; network performs well on training dataset but not on test dataset).

Various methods and criteria can be used to measure performance of a neural network model. For example, root mean squared error (RMSE) measures the average distance between observed values and model predictions. Coefficient of Determination (R²) measures correlation (not accuracy) between observed and predicted outcomes. This method may not be reliable if the data has a large variance. Other performance measures include irreducible noise, model bias, and model variance. A high model bias for a model indicates that the model is not able to capture true relationship between predictors and the outcome. Model variance may indicate whether a model is stable (a slight perturbation in the data will significantly change the model fit). The neural network can receive inputs, e.g., vectors, that can be used to generate models that can be used with provider matching, risk model processing, or both, as described herein.

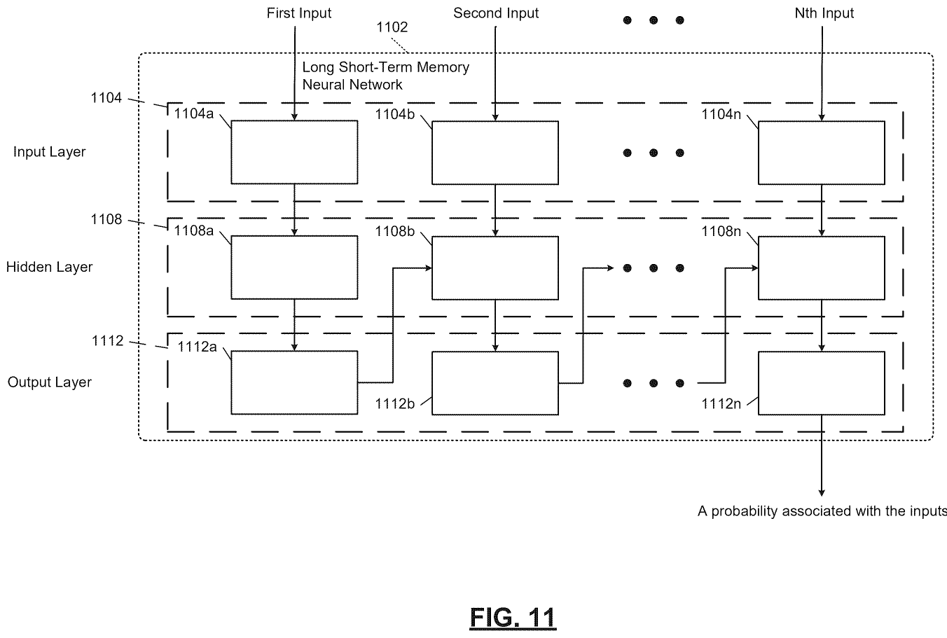

FIG. 11 illustrates an example of a long short-term memory (LSTM) neural network 1102 used to generate models such as those described above, using machine learning techniques. The generic example LSTM neural network 1102 may be used to implement a machine learning model, and various implementations may use other types of machine learning networks. The LSTM neural network 1102 includes an input layer 1104, a hidden layer 1108, and an output layer 1112. The input layer 1104 includes inputs 1104a, 1104b . . . 1104 n. The hidden layer 1108 includes neurons 1108a, 1108b . . . 1108 n. The output layer 1112 includes outputs 1112a, 1112b . . . 1112 n.

Each neuron of the hidden layer 1108 receives an input from the input layer 1104 and outputs a value to the corresponding output in the output layer 1112. For example, the neuron 1108a receives an input from the input 1104a and outputs a value to the output 1112a. Each neuron, other than the neuron 1108a, also receives an output of a previous neuron as an input. For example, the neuron 1108b receives inputs from the input 1104b and the output 1112a. In this way the output of each neuron is fed forward to the next neuron in the hidden layer 1108. The last output 1112n in the output layer 1112 outputs a probability associated with the inputs 1104a-1104n. Although the input layer 1104, the hidden layer 1108, and the output layer 1112 are depicted as each including three elements, each layer may contain any number of elements.

In various implementations, each layer of the LSTM neural network 1102 must include the same number of elements as each of the other layers of the LSTM neural network 1102. In some embodiments, a convolutional neural network may be implemented. Similar to LSTM neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 404a is connected to each of neurons 1108a, 1108b . . . 1108n.

In various implementations, each input node in the input layer may be associated with a numerical value, which can be any real number. In each layer, each connection that departs from an input node has a weight associated with it, which can also be any real number. In the input layer, the number of neurons equals number of features (columns) in a dataset. The output layer may have multiple continuous outputs.

As mentioned above, the layers between the input and output layers are hidden layers. The number of hidden layers can be one or more (one hidden layer may be sufficient for many applications). A neural network with no hidden layers can represent linear separable functions or decisions. A neural network with one hidden layer can perform continuous mapping from one finite space to another. A neural network with two hidden layers can approximate any smooth mapping to any accuracy. The neural network of FIG. 11 can receive inputs, e.g., vectors, that can be used to generate models that can be used with provider matching, risk model processing, or both, as described herein.

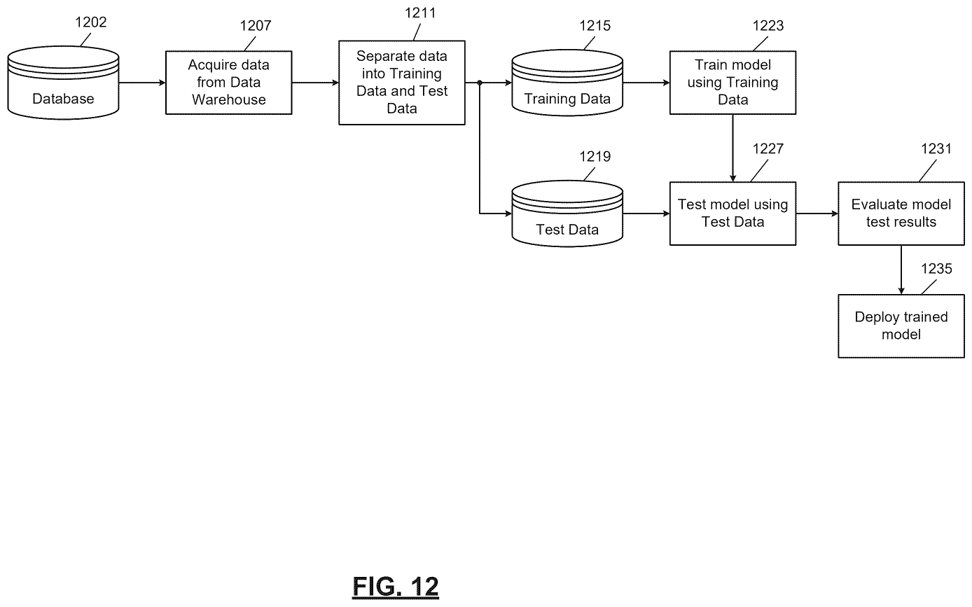

FIG. 12 illustrates an example process for generating a machine learning model. At 1207, control obtains data from a database 1202 (e.g., a data warehouse), such as the database 402. The data may include any suitable data for developing machine learning models.

At 1211, control separates the data obtained from the database 1202 into training data 1215 and test data 1219. The training data 1215 is used to train the model at 1223, and the test data 1219 is used to test the model at 1227. Typically, the set of training data 1215 is selected to be larger than the set of test data 1219, depending on the desired model development parameters. For example, the training data 1215 may include about seventy percent of the data acquired from the database 1202, about eighty percent of the data, about ninety percent, etc. The remaining thirty percent, twenty percent, or ten percent, is then used as the test data 1219.

Separating a portion of the acquired data as test data 1219 allows for testing of the trained model against actual output data, to facilitate more accurate training and development of the model at 1223 and 1227. The model may be trained at 1223 using any suitable machine learning model techniques, including those described herein, such as random forest, generalized linear models, decision tree, and neural networks.

At 1231, control evaluates the model test results. For example, the trained model may be tested at 1227 using the test data 1219, and the results of the output data from the tested model may be compared to actual outputs of the test data 1219, to determine a level of accuracy. The model results may be evaluated using any suitable machine learning model analysis, such as the example techniques described further below.

After evaluating the model test results at 1231, the model may be deployed at 1235 if the model test results are satisfactory. Deploying the model may include using the model to make predictions for a large-scale input dataset with unknown outputs. If the evaluation of the model test results at 1231 is unsatisfactory, the model may be developed further using different parameters, using different modeling techniques, using other model types, etc. The machine learning model method of FIG. 12 can receive inputs, e.g., vectors, that can be used to generate models that can be used with provider matching, risk model processing, or both, as described herein.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements.

The phrase “at least one of A, B, and C” should be construed to mean a logical (A OR B OR C), using a non-exclusive logical OR, and should not be construed to mean “at least one of A, at least one of B, and at least one of C.” The term “set” does not necessarily exclude the empty set. The term “non-empty set” may be used to indicate exclusion of the empty set. The term “subset” does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. Such apparatuses and methods may be described as computerized apparatuses and computerized methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A computer-implemented method for controlling processing of healthcare data within a distributed computing system, the method comprising:
storing, in one or more memory devices, a plurality of patient-specific records including patient data, claims data, and prescription data, and storing provider data including provider feature factors and corresponding weights in a structured memory table;
generating, by a processor, for each patient in a set of patient records, a time-ordered sequence of provider interaction records based on longitudinal engagement between the patient and multiple providers;
transforming the time-ordered sequence into a fixed-length feature representation including a plurality of feature vectors stored in memory;
routing, by the processor, the patient-specific records through a multi-stage data processing pipeline including:
an eligibility filtering stage configured to selectively load a subset of the patient-specific records into a low-latency processing queue based on evaluation of eligibility criteria applied to the stored patient data, claims data, and prescription data; and
an exclusion filtering stage configured to dynamically remove records from the low-latency processing queue based on exclusion criteria, thereby reducing a volume of records processed in subsequent stages;
executing, by the processor, a trained recurrent neural network model stored in memory to generate a provider match prediction for each patient-specific record remaining in the low-latency processing queue, wherein:
the trained recurrent neural network model including input nodes corresponding to elements of the fixed-length feature representation and intermediate nodes stored in memory;
execution of the trained recurrent neural network model includes accessing the structured memory table to apply weighted provider feature factors to the feature representation; and
intermediate nodes of the trained recurrent neural network model are dynamically pruned by deallocating associated memory resources for nodes having weights below a threshold, thereby reducing memory usage and computational load during model execution;
generating, by the processor, for each patient-specific record, a provider match output based on the provider match prediction;
automatically updating, by the processor, a provider assignment field in an electronic database associated with the patient-specific record based on the provider match output, the updated provider assignment field controlling subsequent routing of healthcare transactions within the distributed computing system; and
transmitting, by the processor, the provider match output to a user computing device for display and receiving user input confirming or modifying the provider assignment, wherein the electronic database is updated based on the received user input.

2. The computer-implemented method of claim 1, wherein transforming the time-ordered sequence into a fixed-length feature representation includes generating a sliding-window tensor generated from the time-ordered sequence of provider interaction records.

3. The computer-implemented method of claim 1, wherein accessing the structured memory table to apply weighted provider feature factors includes mapping provider feature factors to weight values using a hash-based indexing structure.

4. The computer-implemented method of claim 1, wherein the low-latency processing queue is configured to prioritize patient-specific records based on a processing threshold associated with system resource utilization.

5. The computer-implemented method of claim 1, wherein routing through the multi-stage data processing pipeline reduces a number of records processed by the trained recurrent neural network model.

6. The computer-implemented method of claim 1, wherein dynamically pruning intermediate nodes includes releasing memory allocations associated with the intermediate nodes during runtime execution of the trained recurrent neural network model.

7. The computer-implemented method of claim 1, wherein execution of the trained recurrent neural network model reduces processor cycles relative to execution without pruning of intermediate nodes.

8. The computer-implemented method of claim 1, wherein updating the provider assignment field automatically controls routing of electronic prescription transactions to a selected provider system.

9. The computer-implemented method of claim 1, wherein the electronic database is part of a pharmacy management system configured to process prescription fulfillment requests.

10. The computer-implemented method of claim 1, wherein the provider data is updated by querying multiple external data sources via application programming interfaces.

11. A computing system configured to control processing of healthcare data, comprising:
one or more processors; and
one or more memory devices storing instructions that, when executed by the one or more processors, cause the computing system to:
store a plurality of patient-specific records including patient data, claims data, and prescription data, and store provider data including provider feature factors and corresponding weights in a structured memory table;
generate, for each patient, a time-ordered sequence of provider interaction records based on longitudinal engagement between the patient and multiple providers, and transform the time-ordered sequence into a fixed-length feature representation comprising a plurality of feature vectors;
route the patient-specific records through a multi-stage data processing pipeline including:
an eligibility filtering stage configured to selectively load a subset of the patient-specific records into a low-latency processing queue based on evaluation of eligibility criteria; and
an exclusion filtering stage configured to dynamically remove records from the low-latency processing queue based on exclusion criteria;
execute a trained recurrent neural network model stored in the one or more memory devices to generate a provider match prediction for each patient-specific record remaining in the low-latency processing queue, wherein:
the trained recurrent neural network model comprises input nodes corresponding to elements of the fixed-length feature representation and intermediate nodes;
execution of the trained recurrent neural network model includes accessing the structured memory table to apply weighted provider feature factors; and
intermediate nodes are dynamically pruned by deallocating associated memory resources for nodes having weights below a threshold to reduce memory usage and computational load;
generate a provider match output based on the provider match prediction;
automatically update a provider assignment field in an electronic database associated with the patient-specific record based on the provider match output, the updated provider assignment field controlling subsequent routing of healthcare transactions within the computing system; and
transmit the provider match output to a user computing device and receive user input confirming or modifying the provider assignment, and update the electronic database based on the received user input.

12. The system of claim 11, wherein the fixed-length feature representation comprises a sliding-window tensor generated from the time-ordered sequence of provider interaction records.

13. The system of claim 11, wherein the structured memory table comprises indexed entries mapping provider feature factors to weight values using a hash-based indexing structure.

14. The system of claim 11, wherein the low-latency processing queue is configured to prioritize patient-specific records based on a processing threshold associated with system resource utilization.

15. The system of claim 11, wherein routing through the multi-stage data processing pipeline reduces a number of records processed by the trained recurrent neural network model.

16. The system of claim 11, wherein dynamically pruning intermediate nodes includes releasing memory allocations associated with the intermediate nodes during runtime execution of the trained recurrent neural network model.

17. The system of claim 11, wherein execution of the trained recurrent neural network model reduces processor cycles relative to execution without pruning of intermediate nodes.

18. The system of claim 11, wherein updating the provider assignment field automatically controls routing of electronic prescription transactions to a selected provider system and wherein the provider data is updated by querying multiple external data sources via application programming interfaces.

19. The system of claim 11, wherein the electronic database is part of a pharmacy management system configured to process prescription fulfillment requests.

20. A non-transitory computer-readable medium storing instructions that, when executed by one or more processors, cause a computing system to:
store a plurality of patient-specific records including patient data, claims data, and prescription data, and store provider data including provider feature factors and corresponding weights in a structured memory table;
generate, for each patient, a time-ordered sequence of provider interaction records based on longitudinal engagement and transform the time-ordered sequence into a fixed-length feature representation comprising a plurality of feature vectors;
route the patient-specific records through a multi-stage data processing pipeline including:
selectively loading a subset of the patient-specific records into a low-latency processing queue based on eligibility criteria; and
dynamically removing records from the low-latency processing queue based on exclusion criteria;
execute a trained recurrent neural network model to generate a provider match prediction, including:
applying weighted provider feature factors stored in the structured memory table; and
dynamically pruning intermediate nodes by deallocating memory resources for nodes having weights below a threshold to reduce computational load;
generate a provider match output;
automatically update a provider assignment field in an electronic database to control routing of healthcare transactions; and
transmit the provider match output to a user computing device, receive user input, and update the electronic database based on the user input.
